(19) United States
(12) Patent Application Publication
(10) Pub. No.: US 2025/0248924 A1
(43) Pub. Date: Aug. 7, 2025

(54) COMPOUNDS FROM FRUIT SEED EXTRACTS FOR MELANIN REDUCTION IN SKIN

(71) Applicant: Brant Lawrence Kedrowski, Oshkosh, WI (US)

(72) Inventor: Brant Lawrence Kedrowski, Oshkosh, WI (US)

(21) Appl. No.: 19/046,039

(22) Filed: Feb. 5, 2025

Related U.S. Application Data
(60) Provisional application No. 63/549,881, filed on Feb. 5, 2024.

(51) Int. Cl. A61K 8/9789; A61Q 19/02

(52) U.S. Cl. CPC A61K 8/9789; A61K 8/498; A61K 8/602; A61Q 19/02; A61K 2236/333

(57) ABSTRACT

The disclosure provides a method of isolating one or more compounds present in fruit seed extracts that have anti-tyrosinase or whitening activity, compositions having one or more fruit seed extracts that have anti-tyrosinase or whitening (lightening) activity.

### CROSS-REFERENCE TO RELATED APPLICATION

This application claims the benefit of priority to U.S. Provisional Patent Application Ser. No. 63/549,881 entitled “COMPOUNDS FROM FRUIT SEED EXTRACTS FOR MELANIN REDUCTION IN SKIN,” filed Feb. 5, 2024, the disclosure of which is incorporated herein in its entirety by reference.

### BACKGROUND

There is a significant world market for skin whitening and skin tone evening cosmetic agents to reduce hyperpigmentation from age spots, melasma, sun exposure, freckles, and for general complexion lightening. The current market leading product is the synthetic chemical bleaching agent hydroquinone. While effective, the safety of hydroquinone has been called into question. It has been banned in a number of overseas markets and could soon be banned in the US as well. There is strong commercial potential for safe and effective alternatives to hydroquinone.

### SUMMARY

This disclosure provides methods of providing extracts from seed flours of raspberries and other fruits or vegetables or other botanicals having materials that are enriched in certain compounds, e.g., the compound ellagic acid and/or other related compounds including ellagitannins. Ellagic acid and its derivatives have commercial potential for use in topical skin treatments for skin whitening and wrinkle reduction. In one aspect, compositions enriched in ellagic acid, ellagitannin, or derivatives thereof, are useful in cosmetic applications. In one aspect, the compositions include a natural source of ellagic acid (“from raspberry seeds”) which may provide for higher concentration of ellagic acid than current methods, which compositions may also include other compounds, and so result in higher efficacy, e.g., to reduce hyperpigmentation from age spots, melasma, freckles, and for general complexion lightening.

As described herein, skin whitening and skin tone evening agents may be obtained from seed meal extracts. Thus, the extracts may be employed to inhibit, prevent or reduce hyperpigmentation from age spots, melasma, freckles, and for general complexion lightening. In one aspect, the extracts inhibit the enzyme tyrosinase, which is a key catalyst in the production of melanin. The tyrosinase inhibiting ability of extracts from blackberry, blueberry, broccoli, carrot, cranberry, grape (chardonnay and red), pomegranate, pumpkin, radish, raspberry (red and black), tomato, and watermelon was tested and compared to known inhibitors and commercial skin whitening products. Several of the seed extracts compared favorably with these known inhibitors and a few even exceeded their potency at concentrations of 1 mg/mL. The seed meal extracts were also characterized by high performance liquid chromatography with diode array UV-visible detection as well as by Matrix-Assisted Laser Desorption/Ionization Time-of-Flight Mass Spectrometry. Many of the tested fruit seed extracts had high levels of the molecule ellagic acid. Although ellagic acid is highly insoluble, the presence of other compounds in the fruit seed meals may assist in solubilizing ellagic acid to improve its effectiveness in water-based media. Cold pressed seed meal powders were obtained with yields of about 7% to about 18%, powders of blackberry, blueberry, broccoli, carrot, chia, cranberry, grape (Chardonnay and red), pomegranate, pumpkin, radish, raspberry (red and black), tomato or watermelon may be employed. The tyrosinase activity may be monitored by combining the extract with L-DOPA, and also in parallel optionally with a solution of known inhibitor (or blank), and formation of L-dopachrome measured over time by UV absorbance.

In one aspect, the extract includes ellagic acid:

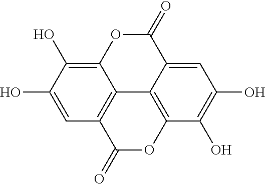 [SMILES: O=c1oc2c(O)c(O)cc3c(=O)oc4c(O)c(O)cc1c4c23]

In one aspect, the extract for use in the compositions and methods does not include one or more of the following: Vitamin E, tocopherols, tocotrinols, carotinods, polyunsaturated fatty acids, sterols, amino acids, or carbohydrates.

The invention thus provides a method to isolate compounds in fruit or fruit seed extracts, such as red raspberry, black raspberry, or blueberry extracts, that have anti-tyrosinase or skin whitening activity, or both, or that include ellagic acid, ellagitannins, or derivatives thereof. The method includes subjecting extracts, e.g., fruit seed extracts such as those from raspberries or grapes, to one or more separation techniques, and optionally identifying, and isolating subfractions with anti-tyrosinase activity, skin whitening activity, ellagic acid or a derivative thereof, or ellagitannins, and optionally repeating separation and identification/isolation steps using different parameters, so as to isolate fractions having a plurality of desired individual compounds, or substantially pure fractions having individual chemical compounds, responsible for anti-tyrosinase or skin whitening activity. In one aspect, the fruit seed extract is a cold press extract which yields small amounts of seed oils and high amounts of seed meal, which are rich in phytochemicals, e.g., 16 pounds of raspberry seeds generally yields 1 teaspoon of oil. In one aspect cold pressed seed meals are dissolved or resuspended so that compounds are extracted into the solvent and the mixture is separated, e.g., compounds in the solvent are collected, and the solvent removed.

In one aspect, a method to isolate compounds having anti-tyrosinase activity is provided. The method includes providing a cold press fruit seed extract dissolved or suspended in water to provide a first mixture. A lower alcohol is combined with the first mixture to provide a second mixture that allows for extraction of compounds including ellagic acid or an ellagitannin. Then the extracted compounds are separated to provide for a fraction that is enhanced in ellagic acid or ellagitannin and has anti-tyrosinase activity.

In one aspect, a method to isolate ellagic acid or ellagitannin from fruit or vegetable seed extracts is provided. In one aspect, the method includes providing a raspberry, blackberry or blueberry seed extract suspended in water, a lower alcohol or a water-lower alcohol mix, thereby providing a mixture. The mixture is incubated under conditions that allow for extraction of compounds including ellagic acid or ellagitannin and the extracted compounds are separated to provide for a fraction that is enhanced in ellagic acid or ellagitannin. In one aspect, the water is at a pH greater than 8.

In one aspect, the lower alcohol is propanol, butanol, or ethanol. In one aspect, the water lower alcohol mixture comprises 70:30 ethanol:water. In one aspect, the lower alcohol is not methanol. In one aspect, the fraction inhibits eumelanin or pheomelanin production in human skin. In one aspect, the extract is a powder. In one aspect, the separation comprises filtering the mixture. In one aspect, the separation includes subjecting the mixture to separation on a C8 column, a C18 solid phase extraction column or an ion exchange chromatography column, or any combination thereof. In one aspect, the separation includes subjecting the mixture to high pressure liquid chromatography. In one aspect, the seed extract is a raspberry, blackberry, blueberry or pomegranate seed extract. In one aspect, a fraction obtained by the method is provided.

Further provided is a use of the fraction in a dietary supplement, nutraceutical, or cosmetic formulation. For example, the fraction may be employed in a formulation such as a lotion, cream, serum, solid or gel for preventing, inhibiting or treating hyperpigmentation. In one aspect, to prepare a pharmaceutical composition, e.g., for topical application, a fruit seed extract is solubilized in a solvent, such as a polar solvent, e.g., ethanol, propanediol or propylene glycol, optionally using agitation. In one aspect, the ratio of solid extract to solvent is about 1:20, e.g., 1:2, 1:5, 1:10 or 1:15. Then undissolved particulates are removed from the solution, e.g., using centrifugation or filtration. Optionally, the solvent, e.g., ethanol, may be removed via evaporation using reduced pressure, and the resulting solid dissolved in a liquid carrier, e.g., ethanol, propanediol or propylene glycol.

In one aspect, to formulate the active ingredient into a cosmetic preparation that diffuses into the skin, the formulation may include a light moisturizing liquid spray using solubilized extract, e.g., solubilized with alcohol, and compatible emollients such as caprylic, capric triglyceride, isopropyl myristate, or other light oils or esters. In one aspect, the formulation may include an oil in water emulsion using propanediol as a carrier added to water phase. In one aspect, the formulation may include liquid or gel serum using polar solvent carrier(s), emollients, and/or viscosity modifier(s). Additional optional ingredients include a suitable preservative system to prevent growth of bacteria, yeasts and/or molds, and/or an antioxidant(s) to prevent oxidation.

Generally, a “substantially pure” composition will comprise more than about 80% of a single macromolecular species present in the composition, e.g., more than about 85%, about 90%, about 95%, and about 99%, and in one aspect, the object species is purified to essential homogeneity (contaminant species cannot be detected in the composition by conventional detection methods) wherein the composition consists essentially of a single macromolecular species. In one aspect, a substantially pure fraction is one where a single compound represents at least 95% of all macromolecular species present in the composition. In one aspect, an isolated fraction is one where a single (first) compound represents at least 50%, or at least 60%, 70%, 80%, 90%, or any integer between 60 and 100, of all macromolecular species in the fraction but other compounds in the fraction individually or in combination with the first compound provide for anti-microbial activity. In one aspect, chromatographic methods were used to separate the multitude of compounds present in fruit seed extracts into several fractions, each containing a simpler mixture of compounds. For example, high performance liquid chromatography (HPLC) and/or solid phase extraction (SPE) may be used to separate these complex mixtures. In one aspect, simulated moving bed chromatography (SMBC) may be employed to separate mixtures. Thus, one type of separation method may be employed once, or two or more different types of separation methods may be employed in succession. In one aspect, one type of chromatography column may be employed individually or in tandem, or two or more different types of columns may be employed sequentially or in tandem. The compounds displaying activity may then be purified in larger quantities, e.g., using the methods to identify those compounds which may include eliminating one or more steps, or through SMBC technology, and the resulting compositions may be employed as a cosmetic, nutraceutical or a pharmaceutical in compositions, e.g., in gel, cream, lotion, powder, tablet or liquid formulations. The structure of the compounds may be determined using proton nuclear magnetic resonance spectroscopy (¹H NMR), MALDI-TOF mass spectrometry, Ultra High Performance Liquid Chromatography (UPLC)-Quadrupole/Time of Flight (Q-TOF) mass spectrometry, and/or ultraviolet (UV) spectroscopy.

Thus, the invention provides compounds, isolated from fruit seed extracts, as well as compositions having that compound, for use in applications including cosmetic applications. In one aspect, the invention provides a composition comprising an isolated fraction of a fruit seed extract having ellagic acid and optionally a pharmaceutically acceptable carrier. In one aspect, the composition is a gel, serum or cream.

### DETAILED DESCRIPTION

### Exemplary Separation Methods

Solid-phase extraction (SPE) is a separation process by which compounds that are dissolved or suspended in a liquid mixture are separated from other compounds in the mixture according to their physical and chemical properties. Analytical laboratories use SPE to concentrate and purify samples for analysis. SPE uses the affinity of solutes dissolved or suspended in a liquid (known as the mobile phase) for a solid through which the sample is passed (known as the stationary phase) to separate a mixture into desired and undesired components. The result is that either the desired analytes of interest or undesired impurities in the sample are retained on the stationary phase. The portion that passes through the stationary phase is collected or discarded, depending on whether it contains the desired analytes or undesired impurities. If the portion retained on the stationary phase includes the desired analytes, they can then be removed from the stationary phase for collection in an additional step, in which the stationary phase is rinsed with an appropriate eluent. The stationary phase comes in the form of, for example, a packed syringe-shaped cartridge, a 96 well plate, a 47- or 90-mm flat disk, or a MEPS device, each of which can be mounted on its specific type of extraction manifold. The manifold allows multiple samples to be processed by holding several SPE media in place and allowing for an equal number of samples to pass through them simultaneously. A typical cartridge SPE manifold can accommodate up to 24 cartridges, while a typical disk SPE manifold can accommodate 6 disks. Most SPE manifolds are equipped with a vacuum port. Application of vacuum speeds up the extraction process by pulling the liquid sample through the stationary phase. The analytes are collected in sample tubes inside or below the manifold after they pass through the stationary phase. Solid phase extraction cartridges and disks are available with a variety of stationary phases, each of which can separate analytes according to different chemical properties. Most stationary phases are based on silica that has been bonded to a specific functional group. Some of these functional groups include hydrocarbon chains of variable length (for reversed phase SPE), quaternary ammonium or amino groups (for anion exchange), and sulfonic acid or carboxyl groups (for cation exchange).

### Reversed Phase SPE

Reversed phase SPE separates analytes based on their polarity. The stationary phase of a reversed phase SPE cartridge is derivatized with hydrocarbon chains, which retain compounds of mid to low polarity due to the hydrophobic effect. The analyte can be eluted by washing the cartridge with a non-polar solvent, which disrupts the interaction of the analyte and the stationary phase.

### Ion Exchange SPE

Ion exchange sorbents separate analytes based on electrostatic interactions between the analyte of interest and the charged groups on the stationary phase. For ion exchange to occur, both the stationary phase and sample must be at a pH where ion-ion interactions may occur.

### Anion Exchange

Anion exchange sorbents are derivatized with positively charged functional groups that interact and retain negatively charged anions, such as deprotonated acids. Strong anion exchange sorbents contain quaternary ammonium groups that have a permanent positive charge in aqueous solutions, and weak anion exchange sorbents use amine groups which are charged when the pH is below about 9. Strong anion exchange sorbents are useful because any strongly acidic impurities in the sample will bind to the sorbent and usually will not be eluted with the analyte of interest; to recover a strong acid a weak anion exchange cartridge should be used. To elute the analyte from either the strong or weak sorbent, the stationary phase is washed with a solvent that neutralizes the charge of either the analyte, the stationary phase, or both. Once the charge is neutralized, the electrostatic interaction between the analyte and the stationary phase no longer exists and the analyte will elute from the cartridge.

### Cation Exchange

Cation exchange sorbents are derivatized with functional groups that interact and retain positively charged cations, such as protonated amines. Strong cation exchange sorbents contain aliphatic sulfonic acid groups that are always negatively charged in aqueous solution, and weak cation exchange sorbents contain aliphatic carboxylic acids, which are charged when the pH is above about 5. Strong cation exchange sorbents are useful because any strongly basic impurities in the sample will bind to the sorbent and usually will not be eluted with the analyte of interest; to recover a strong base a weak cation exchange cartridge should be used. To elute the analyte from either the strong or weak sorbent, the stationary phase is washed with a solvent that neutralizes ionic interaction between the analyte and the stationary phase.

### HPLC

High-performance liquid chromatography (sometimes referred to as high-pressure liquid chromatography), HPLC, is a chromatographic technique used to separate a mixture of compounds in analytical chemistry and biochemistry with the purpose of identifying, quantifying and purifying the individual components of the mixture. Some common examples are the separation and quantitation of performance enhancement drugs (e.g. steroids) in urine samples, or of vitamin D levels in serum. HPLC typically utilizes different types of stationary phases (i.e. sorbents) contained in columns, a pump that moves the mobile phase and sample components through the column, and a detector capable of providing characteristic retention times for the sample components and area counts reflecting the amount of each analyte passing through the detector. The detector may also provide additional information related to the analyte, (e.g., UV/Vis spectroscopic data, if so equipped). Analyte retention time varies depending on the strength of its interactions with the stationary phase, the composition and flow rate of mobile phase used, and on the column dimensions. HPLC is a form of liquid chromatography that utilizes small size columns (typically 250 mm or shorter and 4.6 mm i.d. or smaller; packed with smaller particles), and higher mobile phase pressures compared to ordinary liquid chromatography. With HPLC, a pump (rather than gravity) provides the higher pressure required to move the mobile phase and sample components through the densely packed column. The increased density arises from the use of smaller sorbent particles. Such particles are capable of providing better separation on columns of shorter length when compared to ordinary column chromatography.

The sample to be separated and analyzed is introduced, in a discrete small volume, into the stream of mobile phase being pushed through the column by the pump. The components of the sample move through the column at different velocities, which are functions of specific physical or chemical interactions with the stationary phase. The velocity of each component depends on its chemical nature, on the nature of the stationary phase (column) and on the composition of the mobile phase. The time at which a specific analyte elutes (emerges from the column) is called the retention time. The retention time measured under particular conditions is considered an identifying characteristic of a given analyte. The use of smaller particle size packing materials require the use of higher operational pressure (“backpressure”) and typically improves chromatographic resolution (i.e. the degree of separation between consecutive analytes emerging from the column). Common mobile phases used include any miscible combination of water with various organic solvents (the most common are acetonitrile and methanol). Some HPLC techniques use water free mobile phases (see Normal Phase HPLC below). The aqueous component of the mobile phase may contain buffers, acids (such as formic, phosphoric or trifluoroacetic acid) or salts to assist in the separation of the sample components. The composition of the mobile phase may be kept constant (“isocratic elution mode”) or varied (“gradient elution mode”) during the chromatographic analysis. Isocratic elution is typically effective in the separation of sample components that are not very dissimilar in their affinity for the stationary phase.

In gradient elution the composition of the mobile phase is varied typically from low to high eluting strength. The eluting strength of the mobile phase is reflected by analyte retention times with high eluting strength producing fast elution (=short retention times). A typical gradient profile in reversed phase chromatography might start at 5% acetonitrile (in water or aqueous buffer) and progress linearly to 95% acetonitrile over 5-25 minutes. A period of constant mobile phase composition may be part of any gradient profile. For example, the mobile phase composition may be kept constant at 5% acetonitrile for 1-3 minutes, followed by a linear change up to 95% acetonitrile.

The composition of the mobile phase depends on the intensity of interactions between analytes and stationary phase (e.g. hydrophobic interactions in reversed-phase HPLC). Depending on their affinity for the stationary and mobile phases, analytes partition between the two during the separation process taking place in the column. This partitioning process is similar to that which occurs during a liquid-liquid extraction but is continuous, not step-wise. In this example, using a water/acetonitrile gradient, more hydrophobic components will elute (come off the column) late, once the mobile phase gets more concentrated in acetonitrile (i.e. in a mobile phase of higher eluting strength).

The choice of mobile phase components, additives (such as salts or acids) and gradient conditions depend on the nature of the column and sample components. Often a series of trial runs are performed with the sample in order to find the HPLC method that gives the best separation.

### Partition Chromatography

The partition coefficient principle has been applied in paper chromatography, thin layer chromatography, gas phase and liquid-liquid applications. Partition chromatography uses a retained solvent, on the surface or within the grains or fibers of an “inert” solid supporting matrix as with paper chromatography; or takes advantage of some coulombic and/or hydrogen donor interaction with the solid support. Molecules equilibrate (partition) between a liquid stationary phase and the eluent. Known as Hydrophilic Interaction Chromatography (HILIC) in HPLC, this method separates analytes based on polar differences. HILIC most often uses a bonded polar stationary phase and water miscible, high organic concentration, mobile phases. Partition HPLC has been used historically on unbonded silica or alumina supports. Each works effectively for separating analytes by relative polar differences. HILIC bonded phases have the advantage of separating acidic, basic and neutral solutes in a single chromatogram.

The polar analytes diffuse into a stationary water layer associated with the polar stationary phase and are thus retained. Retention strengths increase with increased analyte polarity, and the interaction between the polar analyte and the polar stationary phase (relative to the mobile phase) increases the elution time. The interaction strength depends on the functional groups in the analyte molecule which promote partitioning but can also include coulombic (electrostatic) interaction and hydrogen donor capability.

Use of more polar solvents in the mobile phase will decrease the retention time of the analytes, whereas more hydrophobic solvents tend to increase retention times.

### Normal-Phase Chromatography

Normal-phase HPLC (NP-HPLC), or adsorption chromatography, separates analytes based on their affinity for a polar stationary surface such as silica, hence it is based on analyte ability to engage in polar interactions (such as hydrogen-bonding or dipole-dipole type interactions) with the sorbent surface. NP-HPLC uses a non-polar, non-aqueous mobile phase, and works effectively for separating analytes readily soluble in non-polar solvents. The analyte associates with and is retained by the polar stationary phase. Adsorption strengths increase with increased analyte polarity. The interaction strength depends not only on the functional groups present in the structure of the analyte molecule, but also on steric factors. The effect of steric hindrance on interaction strength allows this method to resolve (separate) structural isomers.

The use of more polar solvents in the mobile phase will decrease the retention time of analytes, whereas more hydrophobic solvents tend to induce slower elution (increased retention times). Very polar solvents such as traces of water in the mobile phase tend to adsorb to the solid surface of the stationary phase forming a stationary bound (water) layer which is considered to play an active role in retention. This behavior is somewhat peculiar to normal phase chromatography because it is governed almost exclusively by an adsorptive mechanism (i.e., analytes interact with a solid surface rather than with the solvated layer of a ligand attached to the sorbent surface; see also reversed-phase HPLC below). Adsorption chromatography is still widely used for structural isomer separations in both column and thin-layer chromatography formats on activated (dried) silica or alumina supports.

### Displacement Chromatography

The basic principle of displacement chromatography is: A molecule with a high affinity for the chromatography matrix (the displacer) will compete effectively for binding sites, and thus displace all molecules with lesser affinities. There are distinct differences between displacement and elution chromatography. In elution mode, substances typically emerge from a column in narrow, Gaussian peaks. Wide separation of peaks, e.g., to baseline, is desired in order to achieve maximum purification. The speed at which any component of a mixture travels down the column in elution mode depends on many factors. But for two substances to travel at different speeds, and thereby be resolved, there must be substantial differences in some interaction between the biomolecules and the chromatography matrix. Operating parameters are adjusted to maximize the effect of this difference. In many cases, baseline separation of the peaks can be achieved only with gradient elution and low column loadings. Thus, two drawbacks to elution mode chromatography, especially at the preparative scale, are operational complexity, due to gradient solvent pumping, and low throughput, due to low column loadings. Displacement chromatography has advantages over elution chromatography in that components are resolved into consecutive zones of pure substances rather than “peaks”. Because the process takes advantage of the nonlinearity of the isotherms, a larger column feed can be separated on a given column with the purified components recovered at significantly higher concentrations.

### Reversed-Phase Chromatography (RPC)

Reversed phase HPLC (RP-HPLC) has a non-polar stationary phase and an aqueous, moderately polar mobile phase. One common stationary phase is a silica which has been surface-modified with RMe₂SiCl, where R is a straight chain alkyl group such as C₁₈H₃₇ or C₈H₁₇. With such stationary phases, retention time is longer for molecules that are less polar, while polar molecules elute more readily (early in the analysis). An investigator can increase retention times by adding more water to the mobile phase; thereby making the affinity of the hydrophobic analyte for the hydrophobic stationary phase stronger relative to the now more hydrophilic mobile phase. Similarly, an investigator can decrease retention time by adding more organic solvent to the eluent. RP-HPLC is so commonly used that it is often incorrectly referred to as “HPLC” without further specification. The pharmaceutical industry regularly employs RP-HPLC to qualify drugs before their release.

RP-HPLC operates on the principle of hydrophobic interactions, which originate from the high symmetry in the dipolar water structure and play the most important role in all processes in life science. RP-HPLC allows the measurement of these interactive forces. The binding of the analyte to the stationary phase is proportional to the contact surface area around the non-polar segment of the analyte molecule upon association with the ligand on the stationary phase. This solvophobic effect is dominated by the force of water for “cavity-reduction” around the analyte and the C₁₈-chain versus the complex of both. The energy released in this process is proportional to the surface tension of the eluent (water: 7.3×10⁻⁶ J/cm², methanol: 2.2×10⁻⁶ J/cm²) and to the hydrophobic surface of the analyte and the ligand respectively. The retention can be decreased by adding a less polar solvent (methanol, acetonitrile) into the mobile phase to reduce the surface tension of water. Gradient elution uses this effect by automatically reducing the polarity and the surface tension of the aqueous mobile phase during the course of the analysis.

Structural properties of the analyte molecule play an important role in its retention characteristics. In general, an analyte with a larger hydrophobic surface area (C—H, C—C, and generally non-polar bonds, such as S—S and others) is retained longer because it is non-interacting with the water structure. On the other hand, analytes with higher polar surface area (conferred by the presence of polar groups, such as —OH, —NH₂, COO or —NH₃⁺ in their structure) are less retained as they are better integrated into water. Such interactions are subject to steric effects in that very large molecules may have only restricted access to the pores of the stationary phase, where the interactions with surface ligands (alkyl chains) take place. Such surface hindrance typically results in less retention.

Retention time increases with hydrophobic (non-polar) surface area. Branched chain compounds elute more rapidly than their corresponding linear isomers because the overall surface area is decreased. Similarly, organic compounds with single C—C-bonds elute later than those with a C═C or C—C-triple bond, as the double or triple bond is shorter than a single C—C-bond.

Aside from mobile phase surface tension (organizational strength in eluent structure), other mobile phase modifiers can affect analyte retention. For example, the addition of inorganic salts causes a moderate linear increase in the surface tension of aqueous solutions (ca. 1.5×10⁻⁷ J/cm² per Mol for NaCl, 2.5×10⁻⁷ J/cm² per Mol for (NH₄)₂SO₄), and because the entropy of the analyte-solvent interface is controlled by surface tension, the addition of salts tend to increase the retention time. This technique is used for mild separation and recovery of proteins and protection of their biological activity in protein analysis (hydrophobic interaction chromatography, HIC).

Another important factor is the mobile phase pH since it can change the hydrophobic character of the analyte. For this reason most methods use a buffering agent, such as sodium phosphate, to control the pH. Buffers serve multiple purposes: control of pH, neutralize the charge on the silica surface of the stationary phase and act as ion pairing agents to neutralize analyte charge. Ammonium formate is commonly added in mass spectrometry to improve detection of certain analytes by the formation of analyte-ammonium adducts. A volatile organic acid such as acetic acid, or most commonly formic acid, is often added to the mobile phase if mass spectrometry is used to analyze the column effluent. Trifluoroacetic acid is used infrequently in mass spectrometry applications due to its persistence in the detector and solvent delivery system, but can be effective in improving retention of analytes such as carboxylic acids in applications utilizing other detectors, as it is a fairly strong organic acid. The effects of acids and buffers vary by application but generally improve chromatographic resolution.

### Size-Exclusion Chromatography

Size-exclusion chromatography (SEC), also known as gel permeation chromatography or gel filtration chromatography, separates particles on the basis of size. It is generally a low-resolution chromatography method and thus it is often reserved for the final, “polishing” step of a purification. It is also useful for determining the tertiary structure and quaternary structure of purified proteins. SEC is used primarily for the analysis of large molecules such as proteins or polymers. SEC works by trapping these smaller molecules in the pores of a particle. The larger molecules simply pass by the pores as they are too large to enter the pores. Larger molecules therefore flow through the column quicker than smaller molecules, that is, the smaller the molecule, the longer the retention time.

This technique is widely used for the molecular weight determination of polysaccharides. SEC is the official technique (suggested by European pharmacopeia) for the molecular weight comparison of different commercially available low-molecular weight heparins.

### Ion-Exchange Chromatography

In ion-exchange chromatography (IC), retention is based on the attraction between solute ions and charged sites bound to the stationary phase. Ions of the same charge are excluded. Types of ion exchangers include: polystyrene resins which allow cross linkage which increases the stability of the chain. Higher cross linkage reduces resin swelling, which increases the equilibration time and ultimately improves selectivity; cellulose and dextran ion exchangers (gels) possess larger pore sizes and low charge densities making them suitable for protein separation; and controlled-pore glass or porous silica. In general, ion exchangers favor the binding of ions of higher charge and smaller radius.

An increase in counter ion (with respect to the functional groups in resins) concentration reduces the retention time. A decrease in pH reduces the retention time in cation exchange while an increase in pH reduces the retention time in anion exchange. By lowering the pH of the solvent in a cation exchange column, for instance, more hydrogen ions are available to compete for positions on the anionic stationary phase, thereby eluting weakly bound cations.

This form of chromatography is widely used in the following applications: water purification, preconcentration of trace components, ligand-exchange chromatography, ion-exchange chromatography of proteins, high-pH anion-exchange chromatography of carbohydrates and oligosaccharides, and others.

### Bioaffinity Chromatography

This chromatographic process relies on the property of biologically active substances to form stable, specific, and reversible complexes. The formation of these complexes involves the participation of common molecular forces such as the Van der Waals interaction, electrostatic interaction, dipole-dipole interaction, hydrophobic interaction, and the hydrogen bond. An efficient, biospecific bond is formed by a simultaneous and concerted action of several of these forces in the complementary binding sites.

### Aqueous Normal-Phase Chromatography

Aqueous normal-phase chromatography (ANP) is a chromatographic technique which encompasses the mobile phase region between reversed-phase chromatography (RP) and organic normal phase chromatography (ONP). This technique is used to achieve unique selectivity for hydrophilic compounds, showing normal phase elution using reverse-phase solvents.

### Isocratic Flow and Gradient Elution

A separation in which the mobile phase composition remains constant throughout the procedure is termed isocratic (meaning constant composition). The mobile phase composition does not have to remain constant. A separation in which the mobile phase composition is changed during the separation process is described as a gradient elution. One example is a gradient starting at 10% methanol and ending at 90% methanol after 20 minutes. The two components of the mobile phase are typically termed “A” and “B”; A is the “weak” solvent which allows the solute to elute only slowly, while B is the “strong” solvent which rapidly elutes the solutes from the column. In reverse-phase chromatography, solvent A is often water or an aqueous buffer, while B is an organic solvent miscible with water, such as acetonitrile, methanol, ethanol, THF, or isopropanol.

In isocratic elution, peak width increases with retention time linearly according to the equation for N, the number of theoretical plates. This leads to the disadvantage that late-eluting peaks get very flat and broad. Their shape and width may keep them from being recognized as peaks.

Gradient elution decreases the retention of the later-eluting components so that they elute faster, giving narrower (and taller) peaks for most components. This also improves the peak shape for tailed peaks, as the increasing concentration of the organic eluent pushes the tailing part of a peak forward. This also increases the peak height (the peak looks “sharper”), which is important in trace analysis. The gradient program may include sudden “step” increases in the percentage of the organic component, or different slopes at different times—all according to the desire for optimum separation in minimum time.

In isocratic elution, the selectivity does not change if the column dimensions (length and inner diameter) change—that is, the peaks elute in the same order. In gradient elution, the elution order may change as the dimensions or flow rate change.

The driving force in reversed phase chromatography originates in the high order of the water structure. The role of the organic component of the mobile phase is to reduce this high order and thus reduce the retarding strength of the aqueous component.

### Parameters

### Internal Diameter

The internal diameter (ID) of an HPLC column is one parameter that influences the detection sensitivity and separation selectivity in gradient elution. It also determines the quantity of analyte that can be loaded onto the column. Larger columns are usually seen in industrial applications, such as the purification of a drug product for later use. Low-ID columns have improved sensitivity and lower solvent consumption at the expense of loading capacity. Larger ID columns (over 10 mm) are used to purify usable amounts of material because of their large loading capacity. Analytical scale columns (4.6 mm) have been the most common type of columns. They are used in traditional quantitative analysis of samples and often use a UV-Vis absorbance detector. Narrow-bore columns (1-2 mm) are used for applications when more sensitivity is desired either with special UV-Vis detectors, fluorescence detection or with other detection methods like liquid chromatography-mass spectrometry Capillary columns (under 0.3 mm) are used almost exclusively with alternative detection means such as mass spectrometry. They are usually made from fused silica capillaries, rather than the stainless steel tubing that larger columns employ.

### Particle Size

Most traditional HPLC is performed with the stationary phase attached to the outside of small spherical silica particles (very small beads). These particles come in a variety of sizes with 5 μm beads being the most common. Smaller particles generally provide more surface area and better separations, but the pressure required for optimum linear velocity increases by the inverse of the particle diameter squared.

This means that changing to particles that are half as big, keeping the size of the column the same, will double the performance, but increase the required pressure by a factor of four. Larger particles are used in preparative HPLC (column diameters 5 cm up to >30 cm) and for non-HPLC applications such as solid-phase extraction.

### Pore Size

Many stationary phases are porous to provide greater surface area. Small pores provide greater surface area while larger pore size has better kinetics, especially for larger analytes. For example, a protein which is only slightly smaller than a pore might enter the pore but does not easily leave once inside.

### Pump Pressure

Pumps vary in pressure capacity, but their performance is measured on their ability to yield a consistent and reproducible flow rate. Pressure may reach as high as 40 MPa (6000 lbf/in2), or about 400 atmospheres. Modern HPLC systems have been improved to work at much higher pressures, and therefore are able to use much smaller particle sizes in the columns (<2 μm). These “Ultra High Performance Liquid Chromatography” systems or RSLC/UHPLCs can work at up to 100 MPa (15,000 lbf/in²), or about 1000 atmospheres. The term “UPLC” is a trademark of the Waters Corporation, but is sometimes used to refer to the more general technique.

In chromatography, the simulated moving bed (SMB) technique is a variant of high performance liquid chromatography; it is used to separate particles and/or chemical compounds that would be difficult or impossible to resolve otherwise. This increased separation is brought about by a valve-and-column arrangement that is used to lengthen the stationary phase indefinitely.

In the simulated moving bed technique of preparative chromatography, the feed entry and the analyte recovery are simultaneous and continuous, but because of practical difficulties with a continuously moving bed in the simulated moving bed technique, instead of moving the bed, the sample inlet and the analyte exit positions are moved continuously, giving the impression of a moving bed.

True moving bed chromatography (MBC) is only a theoretical concept. Its simulation, SMBC is achieved by the use of a multiplicity of columns in series and a complex valve arrangement, which provides for sample and solvent feed, and also analyte and waste takeoff at appropriate locations of any column, whereby it allows switching at regular intervals the sample entry in one direction, the solvent entry in the opposite direction, whilst changing the analyte and waste takeoff positions appropriately as well.

One advantage of the SMBC is high speed, because a system could be near continuous, whilst its disadvantage is that it only separates binary mixtures. It does not say, but perhaps it can be assumed that this is equivalent with the separation of a single component from a group of compounds. With regard to efficiency, it compares with a simple chromatography technique like continuous distillation does with batch distillation.

Specifically, an SMB system has two or more identical columns, which are connected to the mobile phase pump, and each other, by a multi-port valve. The plumbing is configured in such a way that:
a) all columns will be connected in series, regardless of the valve's position;
b) each different position of the valve will reconnect the columns to one another in one possible sequential arrangement of the columns;
and
c) all possible positions of the valve will arrange the columns in every possible sequential order.

For example, consider a case where two HPLC columns, A and B, are connected to one another, and the mobile-phase pump, via a six-port, two-position valve (e.g., a Rheodyne 7000). One valve position will distribute the flow in the manner
Pump-->Column A-->Column B-->Waste,
   while the other position will distribute the flow in the manner
Pump-->Column B-->Column A-->Waste.

Consequently, switching of the valve will “leapfrog” the columns over one another. If elution across two columns in series is not adequate to resolve two compounds in a given run, the eluent can then be made to go through 3, 4, 5 . . . columns in additional runs by carefully timed switching. This increases the number of theoretical plates until separation can be attained.

When affinity differences between molecules are very small, it is sometimes not possible to improve resolution via mobile- or stationary-phase changes. In these cases, the multi-pass approach of SMB can separate mixtures of those compounds by allowing their small retention time differences to accumulate.

At industrial scale an SMB chromatographic separator is operated continuously, requiring less resin and less solvent than batch chromatography. The continuous operation facilitates operation control and integration into production plants.

In size exclusion chromatography, where the separation process is driven by entropy, it is not possible to increase the resolution attained by a column via temperature or solvent gradients. Consequently, these separations often require SMB, to create usable retention time differences between the molecules or particles being resolved. SMB is also very useful in the pharmaceutical industry, where resolution of molecules having different chirality must be done on a very large scale.

### Exemplary Compositions and Methods of Use

In one aspect, the methods are employed to isolate skin whitening compounds in fruit or vegetable seed extracts, compounds including ellagic acid, or derivatives thereof or ellagitannins, or other compounds with anti-tyrosinase activity or skin whitening activity. A composition may comprise a skin whitening or tyrosinase inhibiting compound in an amount of about 1 μg to about 2000 mg of the compound per dose for a mammal weighing about 20 to 25 g. In one aspect, the composition comprises a compound of the invention an amount of about 1 mg to about 1000 mg, e.g., about 10 mg to about 100 mg, or an amount of about 0.1 μg to about 1000 μg, e.g., about 1 μg to about 10 μg. In one aspect, the composition comprises a compound of the invention an amount of about 20 μg/kg to about 2000 μg/kg, e.g., about 50 μg/kg to about 500 μg/kg or about 100 μg/kg to about 400 μg/kg. Other anti-tyrosinase compounds or other skin whitening agents may be included. In addition to the active agent(s), one or more suitable pharmaceutically acceptable carriers may be used. As used herein, the term “pharmaceutically acceptable carrier” refers to an acceptable vehicle for administering a composition to mammals comprising one or more non-toxic excipients that do not react with or reduce the effectiveness of the pharmacologically active agents contained therein. The proportion and type of pharmaceutically acceptable carrier in the composition may vary, depending on the chosen route of administration. Suitable pharmaceutically acceptable carriers for the compositions of the present disclosure are described in the standard pharmaceutical texts. See, e.g., “Remington's Pharmaceutical Sciences”, 18th Ed., Mack Publishing Company, Easton, Pa. (1990). Specific non-limiting examples of suitable pharmaceutically acceptable carriers include water, saline, dextrose, glycerol, ethanol, or the like and combinations thereof. Optionally, the composition may further comprise minor amounts of auxiliary substances such as antimicrobial agents, stabilizers, preservatives, and the like. Examples of suitable stabilizers include sugars such as sucrose and glycerol, encapsulating polymers, chelating agents such as ethylenediaminetetracetic acid (EDTA), proteins and polypeptides such as gelatin and polyglycine and combinations thereof.

Ellagic acid and its derivatives include compounds of formula (I):

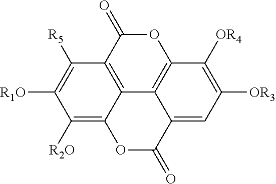

in which R1 to R4 are each, independently, hydrogen atoms, an alkyl group having 1 to 20 carbon atoms (e.g., 1 to 10 carbon atoms, or 1-6 carbon atoms), an alkoxy group having 1 to 20 carbon atoms (e.g., 1 to 10 carbon atoms, or 1-6 carbon atoms), an alkoxy group having 1 to 20 carbon atoms (e.g., 1 to 10 carbon atoms, or 1-6 carbon atoms), a poly(ethylene oxide) or poly(propylene oxide) or poly(ethylene/propylene oxide) (a copolymer of ethylene oxide and propylene oxide units) radical or a sugar radical, and R5 is a hydrogen atom, a hydroxyl group or an alkoxy group having 1 to 18 carbon atoms (e.g., 1 to 10 carbon atoms, or 1-6 carbon atoms). These compounds are described in the patent U.S. Pat. No. 5,073,545, which is hereby incorporated by reference.

### Exemplary Pharmaceutical Formulations

The therapeutic compositions described herein will typically include a carrier (e.g., solvent) and one or more active agents (“actives”), as described above. The compositions can be combined with other ingredients to provide pharmaceutical formulations. Pharmaceutical formulations will typically include the therapeutic composition, a pharmaceutically acceptable carrier, and optionally one or more additional ingredients that, for example, aid the formation of the desired delivery vehicle of the active. For topical administration, it will generally be desirable to administer the active agent to the skin as a composition or formulation, for example, in combination with a dermatologically acceptable carrier, where the composition or formulation may be a semi-solid, oil, or a liquid.

Thus, a topical composition typically includes an active and a pharmaceutically acceptable carrier for topical administration. The administration can be the topical application of a gel, a serum, a jelly, a cream, a lotion, a wax, an ointment, a solution, a paste, an aerosol, a patch, and/or a combination thereof.

Salts. In cases where actives are sufficiently basic or acidic to form stable nontoxic acid or base salts, administration of the actives as salts may be appropriate. Examples of pharmaceutically acceptable salts are organic acid addition salts formed with acids which form a physiological acceptable anion, for example, tosylate, methanesulfonate, acetate, propionate, citrate, malonate, tartrate, succinate, benzoate, ascorbate, α-ketoglutarate, and α-glycerophosphate. Pharmaceutically acceptable salts can also be mineral acid salts such as hydrochlorides, hydrobromides, and the like. Suitable salts may also be formed as halides, nitrates, phosphates, sulfates, bicarbonates, carbonate salts, and the like.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

Carriers. Active agents can be combined with a pharmaceutically acceptable carrier or diluent (e.g., a solvent) to produce a pharmaceutical composition or formulation. In such pharmaceutical formulations, the active agents or therapeutic composition can be combined with a “carrier” that is physiologically compatible with the skin of a human or animal to which it is topically administered. Typically the carrier is substantially inactive, with the exception of its intrinsic surfactant properties which may aid in the production of a solution or suspension of the active ingredients. The compositions may include other physiologically active constituents that do not interfere with the efficacy of the active agents in the composition. In some aspects, the carriers can be liquid or gel-based materials for use in liquid or gel formulations. The specific formulations depend, in part, upon the desired routes or modes of administration.

Suitable carrier materials include any carrier or vehicle commonly used as a base for solutions, dispersions, emulsions, gels, creams, ointment, lotions, pastes, or foams, for topical administration.

Many suitable liquid or gel-based carriers are well-known in the art. The carrier should be able to dissolve or disperse an active at an effective level. Examples include water, physiological salt solutions, alcohols (e.g., ethanol, propanol, or butanol), glycerol, glycols (e.g., ethylene glycol, propylene glycol, or ethoxy diglycol), polyethylene glycol (e.g., MW 400 to 20,000), water-alcohol/glycol blends, and the like. Suitable carriers and diluents for certain aspects include, for example, water, saline, isotonic saline solutions, for example, phosphate-buffered saline, aqueous dextrose, glycerol, ethoxy diglycol, dimethyl sulfoxide (DMSO), and the like, or combinations thereof.

Suitable carriers further include aqueous and oleaginous carriers such as, for example, white petrolatum, isopropyl myristate, lanolin or lanolin alcohols, mineral oil, fragrant or essential oil, nasturtium extract oil, sorbitan mono-oleate, cetostearyl alcohol (together or in various combinations), and detergents (e.g., polysorbates (Tweens) such as polysorbate 20, 40, 60, or 80; polyoxyl stearate; or sodium lauryl sulfate). One or more carrier materials can be mixed with water to form a lotion, gel, cream, semi-solid composition, or the like. Other suitable carriers include water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether, water, or combinations thereof. For example, emulsions containing water, glycerol stearate, glycerin, mineral oil, synthetic spermaceti, cetyl alcohol, or combinations thereof, may be used. Preservatives may also be included in the carrier, such as one or more of butylparaben, methylparaben, propylparaben, benzyl alcohol, and ethylene diamine tetraacetate salts. The composition of the carrier can be varied so long as it does not interfere significantly with the pharmacological activity of the active ingredients of the therapeutic composition.

Gelling Agents and Thickening Agents. The compositions described herein can include one or more gelling agents to increase the viscosity of the composition. Examples of gelling agents and thickening agents, include, but are not limited to, fatty acids, fatty acid salts and esters, fatty alcohols, synthetic polymers, modified celluloses, xanthan gum, or combinations thereof. Examples of suitable synthetic polymers include polyethylene glycol (PEG), polyvinylpyrrolidone (PVP), polyvinyl alcohol (PVA), various Pluronics (poloxamers), or carbomers (e.g., Carbomer 940 or Carbomer 934). Examples of suitable modified celluloses include methylcellulose, carboxymethylcellulose (CMC), hydroxyethylcellulose (HEC), hydroxymethyl cellulose (HMC), hydroxypropyl cellulose (HPC), hydroxypropyl-methylcellulose (HPMC), or other cellulose-based gelling agents.

A variety of gelling agents is commercially available and can be obtained in many suitable molecular weights and ranges. Examples of thickening agents include lanolin, hard paraffin, liquid paraffin, white petrolatum, soft yellow paraffin or soft white paraffin, white beeswax, yellow beeswax, propolis (propoleum), cetostearyl alcohol, cetyl alcohol, dimethicones, emulsifying waxes, microcrystalline wax, oleyl alcohol and stearyl alcohol.

One or more gelling agents or thickening agents may be included in a single formulation. Such agents can be employed with liquid carriers to form spreadable gels, pastes, ointments, soaps, and the like, for application directly to the skin of the user.

Solutions and Dispersions. Solutions of an active or its salts can be prepared in water, optionally mixed with a nontoxic surfactant. Dispersions can be prepared in glycerol, liquid polyethylene glycols, triacetin, or in a pharmaceutically acceptable oil, or mixtures thereof. Under ordinary conditions of storage and use, preparations may contain a preservative to prevent the growth of microorganisms.

Pharmaceutical dosage forms can include sterile aqueous solutions or dispersions comprising the active ingredient adapted for the extemporaneous preparation of sterile solutions or dispersions, optionally encapsulated in liposomes. The ultimate dosage form should be fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, emu oil, nontoxic glyceryl esters, and suitable mixtures thereof. The proper fluidity of the composition can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the compositions can be brought about by agents delaying absorption, for example, aluminum monostearate and/or gelatin.

Solutions can be prepared by incorporating the active in a desired amount in the appropriate solvent or oil with various other ingredients enumerated herein, as desired, followed by optional filter sterilization. For powders used in the preparation of solutions, methods of preparation can include vacuum drying and freeze drying techniques, which yield a powder of the active plus any additional desired ingredient present in the prepared solutions.

Gels. Gels are clear, sticky, jelly-like semisolids or solids prepared from high molecular weight polymers in an aqueous or alcoholic base. Alcoholic gels are often drying and cooling. Non-alcoholic gels are more lubricating. Gels or jellies can be produced using a suitable gelling agent including, but not limited to, gelatin, tragacanth, a carbomer, or a cellulose derivative and may include glycerol as a humectant, an emollient, and/or a preservative. In some aspects, gel formulations will include the same or similar ingredients as a solution or dispersion, with the addition of a gelling agent.

The gel can include a nonionic copolymer gelling agent. In one aspect, the gelling agent is a nonionic polyoxyethylene-polyoxypropylene copolymer gel, for example, a Pluronic gel such as Pluronic F-127 (BASF Corp.), to provide a pluronic gel-based formulation. This gel can be advantageous because it is a liquid at low temperatures but rapidly sets at physiological temperatures, which confines the release of the agent to the site of application or immediately adjacent that site. Other formulations can be carboxymethylcellulose (CMC)-based formulations, hydroxymethyl cellulose (HMC)-based formulations, hydroxypropyl cellulose (HPC)-based formulations, or hydroxypropylmethylcellulose (HPMC)-based formulations, and the like.

Creams. Creams are viscous liquids or semisolid emulsions, either oil-in-water or water-in-oil. Cream bases are water-washable, and comprise an oil phase, an emulsifier, and an aqueous-phase. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant (a substance, such as glycerin, sorbitol, or urea, that absorbs or helps another substance retain moisture).

The emulsifier in a cream formulation is generally a nonionic, anionic, cationic, or amphoteric surfactant. Examples of emulsifiers include, but are not limited to, fatty alcohol polyoxyethylene ether (Peregal A-20), stearates such as polyoxylstearate (Softener SG), glyceryl stearate and pegylated forms of glyceryl stearate such as PEG-5 glyceryl stearate, cetyl alcohol, dithranol, or a combination thereof.

Oil-phase ingredients can include, but are not limited to, dimethicone, dimethiconol, cyclomethicone, diisopropyl adipate, cetyl alcohol, stearyl alcohol, paraffin, petrolatum, almond oil, stearic acid, or a combination thereof. In particular aspects, aqueous ingredients can include, but are not limited to, purified water, glycerol (glycerin), propylene glycol, ethyl paraben, a humectant, or a combination thereof.

In some aspects, the cream further comprises one or more film formers including but not limiting to polyglycerylmethacrylate, acrylates/C10-C30 alkyl acrylate cross-polymers; antioxidant including but not limiting to tocopheryl acetate; preservatives including but not limiting to phenoxyethanol, benzyl alcohol; other additives including but not limiting to dicaprylyl ether, disodium EDTA, sodium hydroxide, and lactic acid.

In one aspect, the cream can include purified water, polyglycerylmethacrylate, propylene glycol, petrolatum, dicaprylyl ether, PEG-5 glyceryl stearate, glycerin, dimethicone, dimethiconol, cetyl alcohol, sweet almond oil, acrylates/C10-C30 alkyl acrylate cross-polymers, tocopheryl acetate, phenoxyethanol, benzyl alcohol, disodium EDT A, sodium hydroxide, lactic acid, or any combination thereof.

In another aspect, the cream can include glycerol, light liquid paraffin, soft white paraffin, dimethicone, squalane, methyl hydroxybenzoate, dichlorobenzyl alcohol, or any combination thereof.

Ointments. Ointments are semisolid preparations that include the active incorporated into a fatty, waxy, or synthetic base. Ointments are typically based on petrolatum or other petroleum derivatives. The specific ointment base to be used, as will be appreciated by those skilled in the art, is one that will provide for suitable drug delivery and other desired characteristics such as emolliency or the like. As with other carriers or vehicles, an ointment base is typically inert, stable, non-irritating and non-sensitizing.

Ointment bases may be generally grouped in four classes: oleaginous bases; emulsifiable bases; emulsion bases; and water-soluble bases. Oleaginous ointment bases can include, for example, vegetable oils, fats obtained from animals, and semisolid hydrocarbons obtained from petroleum. Emulsifiable ointment bases, also known as absorbent ointment bases, contain little or no water and can include, for example, hydroxystearin sulfate, anhydrous lanolin, and hydrophilic petrolatum. Emulsion ointment bases are either water-in-oil (W/O) emulsions or oil-in-water O/W) emulsions, and the oil components can include, for example, cetyl alcohol, glyceryl monostearate, lanolin, and stearic acid. Water-soluble ointment bases can be prepared from polyethylene glycols of varying molecular weight.

Lotions. Lotions are liquid or semiliquid preparations in which solid particles, including the active agent(s), are present in a water or alcohol base. Lotions are usually suspensions of solids, and can include a liquid oily emulsion of the oil-in-water type. Lotions are often desirable formulations because of the ease of applying a more fluid composition. It is generally advantageous for the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Serums. In general, serums have a lower viscosity relative to lotions and gels, and is typified by its rapid absorption, ability to penetrate into the deeper layers of the skin, together with a non-greasy finish and intensive formula with a very high concentration of active substance(s).

Pastes. Pastes are semisolid dosage forms in which the active agent is suspended in a suitable base. Depending on the nature of the base, pastes are divided between fatty pastes or those made from a single-phase aqueous gel. The base in a fatty paste is generally petrolatum, hydrophilic petrolatum, or the like. The pastes made from single-phase aqueous gels generally incorporate carboxymethylcellulose or the like as a base.

Foams. Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Accordingly, the composition described herein may be formulated for any desired form of topical or transdermal administration, including slow or delayed release preparations. Formulations may include known antioxidants (e.g., vitamin E); buffering agents; lubricants (e.g., synthetic or natural beeswax); sunscreens (e.g., para-aminobenzoic acid); and cosmetic agents (e.g., coloring agents, fragrances, essential oils, moisturizers, or drying agents).

An auxiliary agent such as casein, gelatin, albumin, or sodium alginate may also be included in various formulations. Adjuvants such as fragrances and additional antimicrobial agents can be added to optimize the properties for a given use.

Thus, the present disclosure also provides for the use of compositions having ellagic acid, derivatives thereof, or ellagitannins, in a cosmetic preparation for depigmenting and/or whitening the human skin or the hair of the body or scalp. It further provides for the use of this composition for the manufacture of a preparation for depigmenting and/or whitening the human skin or the hair of the body or scalp, after applying to the skin or the hair of the body or scalp the composition. The composition may also include other skin whitening agents, e.g., kojic acid, arbutin, or hydroquinone.

The composition may be present in any of the pharmaceutical forms which are normally used for topical application, e.g., aqueous, aqueous-alcoholic or oily solutions, oil-in-water or water-in-oil or multiple emulsions, aqueous or oily gels, liquid, pastelike or solid anhydrous products, oil dispersed in an aqueous phase with the aid of spherules, it being possible for these spherules to be polymeric nanocapsules or more for example, lipid vesicles of ionic and/or nonionic type.

Moreover, it may be advantageous to dissolve the ellagic acid in a basic solution with a pH greater than 14, then to acidify this solution to a pH of between approximately 6 and 8 prior to its incorporation into the composition.

The present composition may be more or less fluid and may have the appearance of a white or colored cream, a pomade, a milk, a lotion, a serum, a paste or a mousse. The composition can also be applied in aerosol form to the skin or hair of the body or scalp. Additionally, the present composition can be present in solid form and, for example, in stick form. It may be used as a care product and/or as a makeup product for the skin. It may also be in the form of shampoo or conditioner.

Conventionally, the composition may also comprise one or more adjuvants which are customary in the cosmetic field, such as hydrophilic or lipophilic gelling agents, hydrophilic or lipophilic active principles, preservatives, antioxidants, solvents, fragrances, fillers, filters, pigments, odor absorbers and colorants. The amounts of these various adjuvants are those which are conventionally used in the field in question, and, for example, are from 0.01 to 20% of the total weight of the composition. These adjuvants, depending on their nature, may be introduced into the fatty phase, into the aqueous phase, into the lipid vesicles and/or into the nanoparticles. In any case, these adjuvants and their proportions will be selected so as not to detract from the desired properties of the combination of depigmenting agents according to the invention. Such adjuvant selection is well within the ability of one with ordinary skill in the art.

When the composition is an emulsion, the proportion of the fatty phase for example ranges from 5 to 80% by weight, or from 5 to 50% by weight, relative to the total weight of the composition. The oils, emulsifiers and coemulsifiers used in the composition in emulsion form are selected from those which are conventionally used. The emulsifier and coemulsifier are present in the composition in a proportion ranging from 0.3 to 30% by weight, e.g., from 0.5 to 20% by weight, relative to the total weight of the composition.

Suitable oils which may be used include mineral oils (liquid petroleum), oils of vegetable origin (avocado oil, soya oil, sunflower oil, or cranberry oil), black cumin oil, raspberry oil, oils of animal origin (lanolin), synthetic oils (perhydrosqualene), and silicone oils (cyclomethicone). Fatty alcohols (cetyl alcohol), fatty acids, and waxes (camauba wax, ozokerite) may also be used as fatty substances.

Suitable emulsifiers and coemulsifiers which may be used include fatty acid polyethylene glycol esters such as PEG-20 stearate and fatty acid glycerol esters such as glyceryl stearate.

Suitable hydrophilic gelling agents which may be used include carboxyvinyl polymers (carbomer), acrylic copolymers such as acrylate/alkyl acrylate copolymers, polyacrylamides, polysaccharides, natural gums and clays, and, as lipophilic gelling agents, mention may be made of modified clays such as bentones, metal salts of fatty acids, hydrophobic silica and polyethylenes.

The depigmenting or lightening composition is applied in accordance with conventional regimens of depigmentation treatment.

### Exemplary Aspects

In one aspect, the disclosure provides a method to isolate compounds having anti-tyrosinase activity. The method includes providing a cold press processed fruit seed extract dissolved or suspended in water, a lower alcohol or a water-lower alcohol mix to provide a first mixture having solids and a liquid having compounds including ellagic acid or an ellagitannin; and separating the liquid from the solids to provide for a fraction that is enhanced in ellagic acid or ellagitannin and has anti-tyrosinase activity. In one aspect, the method provides for isolation of ellagic acid or ellagitannin. The method includes providing a raspberry, blackberry or blueberry seed extract suspended in water, a lower alcohol or a water-lower alcohol mix, thereby providing a mixture, incubating the mixture under conditions that allow for extraction of compounds including ellagic acid or ellagitannin; and separating the extracted compounds to provide for a fraction that is enhanced in ellagic acid or ellagitannin. In one aspect, the lower alcohol is methanol, ethanol, propanol, or butanol. In one aspect, the water-lower alcohol mix comprises 70:30 ethanol:water. In one aspect, the fraction inhibits eumelanin or pheomelanin production in human skin. In one aspect, the extract is a powder. In one aspect, the extract powder is mixed with methanol and filtered to provide a solution of actives. In one aspect, the extract powder is mixed with a 1:1 mixture of ethanol:acetone and filtered to provide a solution of actives. In one aspect, the method further comprises removing the solvent from the extracted compounds. In one aspect, the separation comprises filtering the mixture. In one aspect, the separation further includes subjecting the mixture to separation on a C8 column, to separation on a C18 solid phase extraction column or to separation on an ion exchange chromatography column, or any combination thereof. In one aspect, the separation further includes subjecting the mixture to high pressure liquid chromatography.

In one aspect, a topical composition comprising an extract of raspberry seed, blackberry seed, blueberry seed or pomegranate seed having ellagic acid or ellagitannin is provided. In one aspect, the composition further comprises a solvent, e.g., an alcohol, oil or water. In one aspect, the composition is a lotion, serum, cream, solid or gel. In one aspect, the extract is a raspberry seed extract. In one aspect, the composition has about 10 wt % to about 30 wt % of the extract. In one aspect, the composition has about 10 wt % to about 90 wt % of the extract. In one aspect, the composition has about 5 wt % to about 20 wt % of the extract. In one aspect, the composition further comprises an emollient. In one aspect, the emollient is about 5 wt % to about 30 wt %, 10 wt % to about 30 wt %, 5 wt % to about 10 wt % or 1 wt % to about 5 wt % of the composition. In one aspect, the composition further comprises a thickener. In one aspect, the composition further comprises a preservative. In one aspect, the thickener is about 0.05 wt % to about 2 wt %. In one aspect, the composition further comprises one or more emulsifiers. In one aspect, the emulsifier is about 1 wt % to about 5 wt % of the composition. In one aspect, the composition further comprises a buffer.

In one aspect, a topical liquid composition comprising an amount of an extract of raspberry seed, blackberry seed, blueberry seed or pomegranate seed having ellagic acid or ellagitannin effective to lighten human skin and a solvent is provided. In one aspect, the extract is a raspberry seed extract. In one aspect, the composition has about 10 wt % to about 30 wt %, about 5 wt % to about 20 wt %, or about 15 wt % to about 35 wt % of the extract. In one aspect, the composition further comprises an emollient. In one aspect, the emollient comprises about 10 wt % to about 30 wt %, about 5 wt % to about 20 wt %, or about 15 wt % to about 35 wt %. In one aspect, the extract is a raspberry seed extract comprising about 10 wt % to about 30 wt and the solvent comprises ethanol.

In one aspect, a topical serum composition comprising an amount of an extract of raspberry seed, blackberry seed, blueberry seed or pomegranate seed having ellagic acid or ellagitannin effective to lighten human skin and a solvent. In one aspect, the extract is a raspberry seed extract. In one aspect, the composition has about 10 wt % to about 30 wt %, 20 wt % to about 50 wt %, 50 wt % to about 90 wt %, or about 10 wt % to about 90 wt % of the extract. In one aspect, the composition of further comprises an emollient. In one aspect, the emollient comprises about 5 wt % to about 30 wt %, about 5 wt % to about 10 wt %, or about 15 wt % to about 35 wt % of the composition. In one aspect, the extract is a raspberry seed extract comprising about 10 wt % to about 90 wt % and the solvent comprises oil. In one aspect, the composition further comprises an emollient at about 5 wt % to about 20 wt %, 5 wt % to about 30 wt % or about 10 wt % to about 40 wt %. In one aspect, the extract is a raspberry seed extract comprising about 10 wt % to about 30 wt % or 5 wt % to about 30 wt % and the solvent comprises water. In one aspect, the composition further comprises one or more emulsifiers. In one aspect, the emulsifier comprises about 1 wt % to about 5 wt %, about 2 wt % to about 6 wt %, or about 3 wt % to about 7 wt %.

In one aspect, a topical oil in water emulsion composition comprising an amount of an extract of raspberry seed, blackberry seed, blueberry seed or pomegranate seed having ellagic acid or ellagitannin effective to lighten human skin and a solvent is provided. In one aspect, the extract is a raspberry seed extract. In one aspect, the composition has about 10 wt % to about 30 wt %, or about 5 wt % to about 20 wt % of the extract. In one aspect, the composition further comprises one or more emulsifiers. In one aspect, the one or more emulsifiers comprise about 2 wt % to about 5 wt %, about 4 wt % to about 10 wt %, or about 5 wt % to about 15 wt % of the composition. In one aspect, the composition further comprises an emollient, a buffer, or both. In one aspect, the solvent comprises water.

The invention will be further described by the following non-limiting examples.

### Example I

In one aspect, the cold press process uses only mechanical pressure to crack and rupture oleosomes, micro sized capsules in the seed, which contain lipids or oil soluble compounds. When the oleosomes are ruptured, the liquid oils flow away from the solid seed material. In one aspect, no solvents or other additives are used in the cold press process. When only mechanical pressure is used, the lipids or oils are extracted from the seed at roughly room temperature and always less than 100° F. In one aspect, no external heat or other materials are used. The cold press process creates two product flows, the oil and residual seed fiber or seed meal. Since the lipids make up a smaller portion by weight of the seed, the remaining seed meal or seed fiber make up the majority of material produced in the cold press process. The range of seed oil content, oil yield and seed fiber yield from red raspberry, black raspberry, red and blackberry is as follows:

| Seed | Oil Content | Oil Yield rate | Meal Yield Rate |
|---|---|---|---|
| Blackberry | 12-15% | 10-11.5% | 84-85% |
| Black Raspberry | 15-17% | 11-12.5% | 83-85% |
| Red Raspberry | 11-15% | 7-12.5% | 83-85% |

The fatty acids of the raw unrefined cold extracted lipids are as follows:

Fatty Acid Analysis Cold pressed Seed Oils

| | Black Raspberry | Red Raspberry | Black Berry |
|---|---|---|---|
| Saturated fats | 5% | 5% | 5% |
| Oleic | 11% | 12% | 14% |
| Linoleic | 53% | 53% | 58% |
| Linolenic | 30% | 29% | 15% |

Nutrition Profile Seed Meals

| | Black | Red | Black | |
|---|---|---|---|---|
| | Raspberry | Raspberry | Berry | Units |
| Calories | 394 | 401 | 411 | calories/100/g |
| Total Fat | 4.25 | 5.4 | 5.8 | % |
| Sodium | 9.1 | 6.7 | 3.1 | mg/100 g |
| Total Carbs | 79 | 78 | 78 | % |
| Total Dietary Fiber | 72 | 71 | 73 | % |
| Protein | 10 | 10 | 12 | % |
| Total Carotenes | 68.3 | | 41 | IU/100 g |
| ‘Calcium | 306 | 206 | 179 | mg/100 g |
| Iron | 9.3 | 6.9 | 7.4 | mg/100 g |
| Moisture | 4.7 | 4.7 | 2.5 | % |

Exemplary ellagic acid content in seed meals was:

| | Total Ellagic Acid (mg/g) |
|---|---|
| Black Raspberry Flour | 14.15 |
| Red Raspberry Seed Flour | 10.01 |
| Pomegranate Seed Flour | .09 |

The above results for ellagic acid content in the seed meal were noteworthy because they were significantly higher than what has been reported in scientific literature for the concentration of ellagic acid in the fruit, e.g., ellagic acid content for red raspberry fruit at 0.024 mg/g. The cold press derived seed meal has an ellagic acid concentration more than 400 times greater than the fruit.

A 2.5 gram sample of a red raspberry seed extract in a dry powder form was split into two fractions, one combined 1.25 grams of the dry extract with 11.25 grams of water to create a 10% aqueous solution of the extract. The extract did not completely go into solution in water as some portion was not soluble. Combining 1.25 grams of the dry extract with about 11.25 grams of isopropyl alcohol (91%) produced an about 10% alcohol solution. This resulted in what appeared to be a near completely dissolved solution.

### Example II—Separation of Compounds from Powder

C18 Solid Phase Extraction. The resuspended powder may be loaded on a C18 solid phase extraction column (Extract-Clean brand, standard C18, 50 μm particle size, 60 angstrom pore size, Grace Davidson Discovery Sciences). The column will be washed with water followed by elution with 15% ethanol, 25% ethanol, and 100% ethanol. Fractions will be analyzed using analytical HPLC

Strong Cation Exchange Chromatography. Fractions may be dissolved in a solution of ethanol and water and loaded onto a strong cation exchange column (Redi-Sep Rf SCX brand, silica based, 40-63 μm particle size, 100 angstrom pore size, Isco-Teledyne) and eluted with a mixture of ethanol and water. Elution is monitored by UV and will proceed until the signal, e.g., at 340 nm, decreases to zero. Analytical HPLC chromatograms may be conducted using diode array UV detection from 250-600 nm.

Preparative Scale High Performance Liquid Chromatography. To further separate components, prep HPLC may be employed. The column may be a Waters Symmetry brand, C8 phase, with 7 μm particle size, and dimensions of 19×150 mm, operated at a flow rate of 12 mL/min. A binary solvent gradient will be used for this HPLC separation with solvent A=water+0.1% formic acid and solvent B=ethanol+0.1% formic acid. Solvent composition at various times will be as follows, time (percent B):initial (10% B), 50 min. (35% B), 80 min. (60% B), 85 min. (80% B), 90 min. (80% B). Individual runs may contain a fraction dissolved in 1:1 water/ethanol to a volume of 1 mL. The progress of the separation will be followed using UV detection, e.g., detection at 255 and 340 nm. Eluent will be collected in tubes at 2 minute intervals and the contents of those tubes pooled according to their UV profile. Evaporation of the solvent from these fractions may yield dry powders. Analytical HPLC chromatograms are conducted with a diode array UV from 250-600 nm.

Analytical Scale High Performance Liquid Chromatography. As a final purification step, fractions may be separated by analytical HPLC.

### Example III—Compounds from Seed Powder

The powder will be mixed with 70% ethanol/30% water solution (100 mL) for 30 minutes. Then Celite (1 g) will be stirred in and the mixture was vacuum filtered through a pad of Celite. Solvents will be removed under vacuum. This material is separated using preparative HPLC using the following column: Waters Symmetry brand, C8 phase, with 7 μm particle size, and dimensions of 19×150 mm, operated at a flow rate of 12 mL/min. A binary solvent gradient will be used for this HPLC separation with solvent A=water+0.1% formic acid and solvent B=ethanol+0.1% formic acid. Solvent composition at various times is as follows, time (percent B): initial (10% B), 50 min. (35% B), 80 min. (60% B), 85 min. (80% B), 90 min. (80% B). Individual runs may contain about 40 mg of seed extract dissolved as a solution of water/ethanol to a volume of 1 mL. The progress of the separation is followed using UV detector at 255 and 340 nm. Eluent will be collected in tubes at 2 minute intervals and the contents of these tubes are pooled according to their UV profile.

### Example IV—Comparison of Different Seed Powders

### Methods and Materials

### Seed Materials:

Seed powders will be obtained from Botanic Innovations. They will be produced by mechanically pressing seeds in their NatureFRESH Cold Press process, which removes most of the seed oils. The solid seed materials that remain after pressing will be ground to a powder that retains a small percentage of seed oils (around 5-10%), and all of the other compounds in the seeds that are not dissolved in the oils. Seed powders from fruits including cranberry, pomegranate, red raspberry, black raspberry, blackberry, blueberry, and watermelon were studied along with the dried skins of cranberries. Seed powders from vegetables including broccoli, radish, tomato and carrot were also studied.

### Extraction Solvents and Criteria:

The following criteria will be applied when screening extraction protocols to serve as the second level of purification (after cold pressing by Botanic Innovations).
1. The extraction process should use inexpensive and widely accessible solvents. Solvents with low flammability and low toxicity were desirable. Therefore, the solvents used in this round of purification were water, alcohols, and mixtures of these solvents.
2. The method should be amenable to scaling up and optionally not use highly specialized equipment.
3. The extraction process should produce high yields of extracted substances.
4. The protocol should effectively extract the desired target substances, but leave residual undesired oils behind.
5. The mixtures of seed powders and solvents after extraction are filterable so as to isolate the filtrate containing the desired compounds. Solvents that produced mixtures with fine particulates, or that require multiple passes of filtration, or that frequently clogged filtering media should be avoided.
6. It is desirable to remove the solvent by distillation to isolate the mixture as a solid or concentrated liquid residue. Therefore, solvents with boiling points above 100° C. will be avoided.

### Chemical Characterization of Extracts by HPLC:

Seed powder extracts was characterized by high performance liquid chromatography (HPLC). After some experimentation, a method was developed that resolved compounds into peaks in runs requiring 45 minutes. The column was a Waters Symmetry brand with dimensions of 150×3.9 mm. It contained a C8 stationary phase with a 5 μm particle size. A constant flow rate of 1.00 mL/min was used in all experiments. A binary solvent system was used with a gradient elution program. The “A” solvent was deionized water with 0.1% added formic acid. The “B” solvent was pure HPLC grade ethanol. The gradient starts at 2% B solvent, which will be held constant for 5 minutes. The concentration of the B solvent then increases from 2% to 50% between 2 min. and 20 min. It was increased from 50% to 100% B solvent between 20 min. and 25 min., and then held at 100% B solvent for 5 min. before returning back to 2% B solvent over 5 min. Injection volumes were between 5-10 μL of analyte solution. Peaks were detected by UV absorption using a diode array detector. This provided the full UV spectrum of each peak, which was used to confirm the identity of unknown peaks by comparing the unknown's UV spectrum and retention time to authentic standards that were purchased from commercial sources. Materials analyzed for included α-arbutin, β-arbutin, ellagic acid, niacinamide, and raspberry ketone. Retention times of the standards used and their maximum UV absorbing wavelengths (λmax) are listed in Table 1.

TABLE 1
Expected HPLC standard retention times and λₘₐₓ values

| Standard | Retention time (min) | λₘₐₓ (nm) |
|---|---|---|
| α-arbutin | 3.44 | 226, 283 |
| β-arbutin | 3.55 | 226, 283 |
| ellagic acid | 21.87 | 255, 369 (weak) |
| niacinamide | 2.53 | 220, 262 |
| raspberry ketone | 19.38 | 228, 277 |

The use of HPLC with a mass spectrometer detector (LC-MS) was employed.

### Tyrosinase Inhibition Assays:

Extracts were tested for their ability to inhibit the enzyme tyrosinase. This enzyme catalyzes the air oxidation of L-DOPA (colorless) to the compound dopachrome, which is a highly colored intermediate in the production of melanin. The dopachrome molecule absorbs strongly at 475 nm. So, measuring the rate of change of absorption at this wavelength using UV/visible spectroscopy can easily assess the enzyme's activity. The tyrosinase enzyme (from mushroom) was purchased from Sigma-Aldrich as a lyophilized powder with an activity of 5771 units per mg of solid (a unit of activity is defined as the rate at which the enzyme catalyzes the reaction of a standard substrate at a standard concentration). This enzyme (1.0 mg) was dissolved in 0.1 M aqueous pH 7.00 phosphate buffer (28.9 mL) to generate a solution with an activity of 200 units/mg, which was used throughout the assays. The L-DOPA substrate was purchased from TCI America and dissolved in deionized water at a concentration of 7.5 mM. Inhibitors were dissolved in DMSO at various concentrations to test their potency. Assay runs were carried out by pipetting solutions into plastic cuvettes in the following order (typical amounts in parentheses). L-DOPA substrate (100 μL) was added followed by phosphate buffer solution (2.35 mL), and a DMSO solution with dissolved inhibitor or a blank of pure DMSO to act as a control (50 μL). The cuvette was covered with a piece of Parafilm and inverted twice to thoroughly mix the solutions. Next, the enzyme solution was added (500 μL), the cuvette was covered with a piece of Parafilm and inverted twice to thoroughly mix the solutions, and immediately after the cuvette was placed in a UV/visible spectrometer and absorbance was measured at 475 nm every second for 60 seconds. The slope of the line formed by graphing change in absorbance versus time gave the rate in units of AA/second. This rate was converted into units of μmol of L-DOPA consumed per minute (μmol/min).

### Results and Discussion

### Extractions Using Water

Using pure water was explored in extraction experiments with cranberry seed powder. The advantages of using water are essentially zero cost and toxicity. It is also a very polar solvent and will not extract any residual oils (which are very non-polar compounds) from the seed powders. The limitations of using water are that it gives quite low yields in extractions at room temperature and only dissolved the most polar compounds from the seed powders, leaving most of the medium polarity materials behind. It is possible that this could be improved by heating. Filtering mixtures of water and seed powders also proved to be rather difficult. Simply filtering the mixture through inexpensive filter paper by gravity or mild vacuum will be unsuccessful. One solution that worked was mixing in filtering media (diatomaceous earth) and then vacuum filtering through a bed of the same filtering medium. To completely clarify the mixture, an additional treatment with activated carbon and further vacuum filtering through a bed of diatomaceous earth was effective. Given the limitations of water extraction, it was not used extensively. One exception was the use of sodium hydroxide solutions in water to extract pomegranate seed powder, which contained significant amounts of the targeted compound ellagic acid. Ellagic acid is only slightly soluble in water at neutral pH, but at high pH it becomes very water soluble. Therefore, 1 M aqueous sodium hydroxide solution was used to extract this material from pomegranate seed powder. After filtering away the solid seed material the solution was acidified to pH=4 by addition of 1 N HCl. A small amount of solid developed, which was filtered.

### Extractions Using Ethanol

Pure ethanol was used in extraction experiments with cranberry and pomegranate seed powders. Each seed material (100.0 g) was mixed with denatured ethanol (400 mL) for 2 hours, and then filtered. These filtrations proceeded smoothly. Evaporation gave residues that were very oily. To separate the residual seed oil from other compounds of interest the residues were washed with petroleum ether (50 mL). This solvent dissolves the oils and gave other compounds as a precipitate, which were filtered. The yield for cranberry seed material was 0.495 g (0.5%). These solid samples were hygroscopic (water absorbing), and became syrupy on exposure to the air. Adding ethanol (2.5 mL) to each gave a precipitate that was insoluble in all common solvents and a filtrate. The cranberry extract was a very complex mixture with numerous peaks, many of which were overlapping.

Extractions with 70:30 Ethanol/Water

Mixtures of pure water and pure alcohol as extraction solvents were used. One highly effective combination was the use of a solution of 70% ethanol and 30% water (volume to volume). This solvent system seems to have the right polarity to provide good yields of desired materials and no problems with co-extraction of undesirable seed oils. Furthermore, extractions with this solvent system are easily filterable. The protocol used 5.00 g of seed powder, which was suspended in deionized water (70 mL), with stirring. Ethanol was added (30 mL) and the mixture was stirred 16 hours at room temperature. Vacuum filtering the mixture through Whatman #1 filter paper quickly gave a clear solution, which was usually tinted a red or yellow color. These solutions were analyzed directly by HPLC. They were then concentrated under reduced pressure on a rotary evaporator at 40° C. to 25 mL, which removed most of the ethanol and left a solid suspension in water. Evaporating further in a stream of air or by freeze drying will gave the residues listed in Table 2. All of the extracts were hygroscopic and absorbed between 0.6% and 1.3% of their mass in water upon standing at room temperature. This tended to make them a very thick syrupy consistency, which was more pronounced in certain extracts and less pronounced in others as noted in Table 2. Table 2 also lists the mass of each extract and percent mass recovery (yield) based on 5.00 g of starting seed powder, or dried cranberry skins. Yields in this process were excellent compared to previous extractions in either pure water or pure ethanol.

TABLE 2
Expected Fruit Seed Extract Mass,
% Yield, Color, and Morphology

| Seed Source | Extract Mass | % Yield | Color | Morphology |
|---|---|---|---|---|
| Cranberry | 0.65 g | 13% | dark red | viscous liquid |
| Pomegranate | 0.89 g | 18% | yellow/brown | viscous liquid |
| Red Raspberry | 0.42 g | 8.4% | salmon | solid |
| Black | 0.33 g | 6.7% | maroon | solid |
| Raspberry | | | | |
| Blackberry | 0.48 g | 9.6% | dark purple | viscous liquid |
| Watermelon | 0.80 g | 16% | orange | viscous liquid |
| Blueberry | 0.77 g | 15% | dark | sticky solid |
| | | | blue/purple | |
| Cranberry skins | 0.99 g | 20% | red | viscous liquid |

In addition to fruit seed powders, four vegetable seed powders from Botanic Innovations were extracted and studied. As with the fruit seed materials, these vegetable seed powders were extracted using a 70:30 (volume/volume) solution of ethanol and water for 16 h with room temperature stirring. Table 3 lists the materials investigated thus far along with the mass of extract obtained from 5.00 g of seed powder, the percent yield of the extraction, the color of the extract after solvents were removed, and the morphology of solvent-free material.

TABLE 3
Expected Vegetable Seed Extract Mass,
% Yield, Color, and Morphology

| Seed Source | Extract Mass | % Yield | Color | Morphology |
|---|---|---|---|---|
| Broccoli | 0.87 g | 17% | tan | solid |
| Radish | 1.01 g | 20% | tan | solid |
| Tomato | 0.43 g | 8.7% | light yellow | solid |
| Carrot | 0.57 g | 11% | amber | hygroscopic |
| | | | | solid |

The vegetable seed powder extracts were also characterized by HPLC with UV detection.

Expedited Extractions with 70:30 Ethanol/Water

To speed up the preparation process, experiments were carried out using shorter extraction times using red raspberry seed powder as a model. The yields and compositions of these materials were compared to the previous results with 16 hour extraction times. It was found that the two hours of extraction time with 70:30 ethanol/water gave identical results to the longer overnight protocol used previously. The yield and morphology of material generated in two hours matched the 16 hour extraction and there were no perceivable differences in the HPLC chromatograms of these materials. It was also found that the ability of the extracts to inhibit tyrosinase enzyme was identical, which will be discussed in the following section. It is possible that even shorter extraction times may be achievable without a decrease in performance. The efficiency advantage of shorter procedures will be important in large scale production of extracts, which is anticipated in the future.

### Tyrosinase Inhibition Studies:

The extracts in Tables 2 and 3, prepared from 70:30 ethanol/water, were tested for their ability to inhibit tyrosinase at concentrations of 1.0 mg/mL, 0.1 mg/mL, 0.01 mg/mL, and in some cases 0.001 mg/mL. Inhibition data is reported in Table 4. For comparison several other known tyrosinase inhibitors including kojic acid, alpha-arbutin, beta-arbutin, hydroquinone, and quercetin were tested. Kojic acid is often used in tyrosinase inhibition studies as a benchmark inhibitor for comparison with other inhibitors. Also included are beta-arbutin and hydroquinone due to their relevance in skin whitening applications. Additionally, several commercial skin whitening agents were also tested. These included a product called Radian Skin from BASF, and an arbutin material sold by the company Presperse. The reference compound ellagic acid was highly insoluble in the enzyme assay and could not be tested at the same levels as the other inhibitors for that reason. Even highly dilute samples of ellagic acid were not sufficiently soluble. The compound quercetin also had similar solubility limitations, but not as severe. Finally, a mixture of red raspberry and tomato extracts was tested to look for possible synergistic effects in mixtures on two quite different materials. The presence of negative inhibition values is simply a reflection of the error of the test, which is likely around +/−5%.

Percentage inhibition values was calculated by taking the rate of the blank run with no inhibitor minus the rate of the inhibited sample and then dividing that value by the blank rate and multiplying by 100. In equation form this is:

Percentage inhibition=[[blank rate−inhibitor rate]/blank rate]*100

### Example V

Ellagitannins and other hydrolyzable tannins in raspberry seed extract powder were characterized by MALDI-TOF Mass Spectrometry.

### Materials and Methods

### Matrix-Assisted Laser Desorption/Ionization Time-of-Flight Mass Spectrometry

Mass spectra were collected on a Bruker MicroflexLRF MALDI-TOF mass spectrometer (Billerica, MA) equipped with delayed extraction and a N2 laser (337 nm). Spectra were collected in negative reflectron mode using an accelerating voltage of 25.0 kV and a reflectron voltage of 26.5 kV. Spectra are the sum of <3000 shots. Spectra were calibrated with bradykinin (Sigma, St. Louis, MO) as an external standard. 2,5-dihydroxybenzoic acid (Aldrich, Milwaukee, WI) solubilized in ethanol (50 mg/mL) was used as a matrix. Samples were mixed with the matrix solution at volumetric ratios of 1:2 and the mixture (1 μL) and were deposited on a stainless steel target. All results will be reported as a deprotonated mass [M-H]-.

### Results and Discussion

### Characterization of Ellagitannins and Other Oligomeric Hydrolyzable Tannin Distribution by Matrix-Assisted Laser Desorption/Ionization-Time of Flight Mass Spectrometry (MALDI-TOF MS)

MALDI-TOF MS was used to identify the monomeric and oligomeric hydrolysable tannins in the sample. The predicted masses are based on structural features of ellagitannin and gallotannin chemistry described by Tanaka et al. (1985, 1986a and 1986b). Tentative structural assignments of compounds are based on substitutions with glucose, gallagic acid, hexahydroxydiphenoyl (HHDP), gallic acid and dehydrodigalloyl acid (DHDG) (Afaq et al.; Martin et al., 2009). While MALDI-TOF MS is able distinguish molecular weight differences due to degree of polymerization and structural substitutions, it does not have the ability to assign specific stereochemistry to a molecule. Results (will indicate that raspberry seed extract powder contained masses with a similar distribution to those describe by Martin et al. (2009) and Kula et al. (2016).

The results will demonstrate the presence of nine specific ellagitannins in the extract. These include ellagtannins that have:
1) a glucose core with two HHDP (hexahydroxydiphenoyl) groups, which has a mass of 784 amu (atomic mass units),
2) a glucose core with one gallagosyl group and one galloyl group, which has a mass of 934 amu,
3) a glucose core with one gallagosyl group and one HHDP group, which has a mass of 1084 amu,
4) a glucose core with two gallagosyl groups, which has a mass of 1086 amu,
5) two glucose cores with one gallagosyl group and two galloyl groups, which has a mass of 1266 amu,
6) two glucose cores with one gallagosyl group, one galloyl group, and one DHDG (dihydrodigalloyl) group, which has a mass of 1416 amu,
7) two glucose cores with three HHDP groups and one DHDG group, which has a mass amu, of 1568
8) two glucose cores with one gallagosyl group, one HHDP group, one galloyl group, and one DHDG group, which has a mass of 1718 amu, and
9) two glucose cores with one gallagosyl group, one HHDP group, two galloyl groups, and one DHDG group, which has a mass of 1870 amu.

TABLE 5
Matrix-assisted laser desorption/ionization time-of-flight
mass spectrometry (MALDI-TOF MS) of ellagitannins and other
hydrolyzable tannins in raspberry seed extract powder.

| | | | | | | Expected | Observed |
|---|---|---|---|---|---|---|---|
| | | | | | | Mass | Mass |
| | Glucosyl | Gallagosyl | HHDP | Galloyl | DHDG | [M − H] | [M − H] |
| Ellagitannin & | 1 | 0 | 2 | 0 | 0 | 783 | 783 |
| Hydrolyzable | 1 | 1 | 0 | 1 | 0 | 933 | 933 |
| tannins | 1 | 1 | 1 | 0 | 0 | 1083 | 1083 |
| | 1 | 2 | 0 | 0 | 0 | 1085 | 1085 |
| Dimers | 2 | 1 | 0 | 2 | 0 | 1265 | 1265 |
| | 2 | 1 | 0 | 1 | 1 | 1415 | 1415 |
| | 2 | 0 | 3 | 0 | 1 | 1567 | 1567 |
| | 2 | 1 | 1 | 1 | 1 | 1717 | 1717 |
| | 2 | 1 | 1 | 2 | 1 | 1869 | 1869 |

HHDP: Hexahydroxydiphenoyl, DHDG: Dehydrodigalloyl.

### Example VI

A skin safety study was conducted. The initial extracts in aqueous and alcoholic solution were used for preliminary testing to determine whether they may cause irritation or skin sensitization. The test method applies a few drops of the extract to the same spot on the skin once daily. On the following day they skin was observed and evaluated using the following scale for signs of redness irritation. Subjects self report. The aqueous form of the extract was used for two subjects (one male and one female) and the alcoholic form of the extract was used by one subject (a female).

### Dermal Scoring Scale

0 No visible skin reaction
+ Barely perceptible erythema
1+ Mild erythema
2+ Well defined erythema
3+ Erythema and edema
4+ Erythema and edma with vesiculation
The following charts are the scores for a 30 day period. No irritation, redness or skin sensitization was reported.

TABLE 6

| | TEST MATERIAL | | | | | | | | | | | | | | |
|---|---|---|---|---|---|---|---|---|---|---|---|---|---|---|---|
| | SKIN LIGHTENING EXTRACTS | | | | | | | | | | | | | | |
| SUBJECT | DAILY INDUCTION SCORES | | | | | | | | | | | | | | |
| NUMBER | 1 | 2 | 3 | 4 | 5 | 6 | 7 | 8 | 9 | 10 | 11 | 12 | 13 | 14 | 15 |
| 1 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 |
| | TEST MATERIAL | | | | | | | | | | | | | | |
| | SKIN LIGHTENING EXTRACTS | | | | | | | | | | | | | | |
| SUBJECT | DAILY INDUCTION SCORES | | | | | | | | | | | | | | |
| NUMBER | 16 | 17 | 18 | 19 | 20 | 21 | 22 | 23 | 24 | 25 | 26 | 27 | 28 | 29 | 30 |
| 1 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 |

### Example VII

15 Asian or Indian subjects age 35-65 with a desire to lighten their skin, with skin type Fitz III-V, will a formulation having an extract described above (RSE) are followed for 6 weeks. The effect of the formulation on skin lightening was assessed using chromameter assessments and expert visual assessments at Week 0 (baseline), Week 3 and Week 6.

### Chromameter® Assessment of Skin Tone

Instrumental measurements of skin tone will be performed using a Chromameter CR300® (Courage and Khazaka, Germany) on the skin. The measuring head of the CR-300 uses diffuse illumination/0° viewing geometry. A pulsed xenon arc (PXA) lamp inside a mixing chamber provides diffuse, uniform lighting over the 8 mm-diameter specimen area. Only the light reflected perpendicular to the specimen surface is collected by the optical-fiber cable for color analysis.

The instrument measures the amount of light reflected from the skin and quantifies this into a numerical value using the L*a*b* colour scale, where L*(100) equates to total white and L*(0) equates to total black. The instrument was allowed to warm up for 30 minutes prior to use. Readings were taken on the right and left side of the upper cheek, cheek bone area. L* values were recorded and analysed.

### Other Analyses

Testing took place amongst the patients, with application to solar lentigines of the face twice a day for 8 weeks. Pictures were taken before and after 8 weeks and patients fill out a questionnaire regarding patient experience (subjective safety/efficacy/ease of use/adverse events) and overall comments/satisfaction. The study controlled for the location of lesions (example cheek bones, forehead, cheeks, etc.) and sun exposure during the evaluation process.

### Example VIII

Different solvents were tested with raspberry seed extract (RSE). The solubility data shows the relative efficiency of various solvents in solubilizing the solid extract particles. Solvents with higher efficiencies can be employed in a cosmetic formulation so that the active agent will be available to penetrate the skin. The study included a second step in an attempt to solubilize the entire particle. However, since the solution from the initial solubility step does include the active agent, insoluble particles will be filtered away rather than using a second solvent.

In addition, solid raspberry seed extract (0.500 g) was combined with ethanol (5 mL), the mixture was agitated, and a white colored precipitate (0.057 g) was separated from a red solution of ethanol. Evaporating the ethanol under vacuum gave a red foam (0.443 g) that was crushed to a free flowing powder. The white colored precipitate was highly insoluble in typical solvents. The material from the ethanol fraction was highly soluble in both ethanol and DMSO. That material was tested in the enzyme assay in triplicate and found it to be of equivalent potency to the original extract. Thus, the insoluble material can be removed without decreasing activity. Removal of the insoluble portion also will make the extract much more soluble and the active material appears to be less hygroscopic after insoluble materials are removed, which makes handling easier.

A variation of the procedure in the prior paragraph was carried out where solid raspberry seed extract (produced from cold pressed raspberry seed flour by extraction with 70:30 ethanol:water) was combined first with pure ethanol (0.29 mL per gram of raspberry seed flour used) followed by pure acetone (0.29 mL per gram of raspberry seed flour used) and the mixture agitated thoroughly by sonication and stirring. This dissolved the active (red colored) portion of the extract, which contains active tyrosinase enzyme inhibiting materials, and caused inactive materials to precipitate out as a white solid. The inactive solid materials were removed by vacuum filtration through a bed of diatomaceous earth, giving a clear red filtrate containing the dissolved active extract. Solvents were removed under vacuum at 40° C. (by rotary evaporation followed by higher vacuum drying using an oil based mechanical pump) to give a refined extract as a dry red foam. The foam was scraped out of the flask to give a dry, free flowing red powder and stored in a tightly sealed container. The percent yield of the entire process (from cold pressed seed meal, through extraction with 70:30 ethanol:water, and redissolving using the protocol in this paragraph was 4.6%. The material was highly soluble in both ethanol and DMSO.

### Example IX

A method was developed to produce an extract of red raspberry seed flour as a dry free flowing powder. The procedure does not utilize methanol. The procedure is described below.
1. A mixture of 200 proof ethanol/deionized water (70:30 by volume) was prepared.
2. Raspberry seed flour (0.25 grams per milliliter of the ethanol/water solution) was added to the mixture.
3. The mixture was covered and stirred overnight.
4. The mixture was filtered through cotton wool and cheese cloth to give a clear red solution.
5. All solvent was completely removed under vacuum at 40° C. (by rotary evaporation) to give a crude extract.
6. Pure ethanol was added to the crude extract (0.29 mL per gram of raspberry seed flour used in step 2) followed by pure acetone (0.29 mL per gram of raspberry seed flour used in step 2) and agitate thoroughly by sonication and stirring. This dissolves the active (red colored) portion of the extract, which contains active tyrosinase enzyme inhibiting materials, and causes inactive materials to precipitate out as a white solid.
7. Inactive solid materials were removed by vacuum filtration through a bed of diatomaceous earth, which gave a clear red filtrate containing the dissolved active extract.
8. Solvent was removed under vacuum at 40° C. (by rotary evaporation followed by higher vacuum drying using an oil based mechanical pump) to give a refined extract as a dry red foam.
9. The foam was scraped out of the flask to give a dry, free flowing red powder and store in a tightly sealed container.
10. The percent yield was

$$4.6 \% = \frac{mass of refined extract}{mass of starting raspberry flour} \times 100.$$

### Exemplary Aspects.

The following exemplary aspects are provided, the numbering of which is not to be construed as designating levels of importance:
Aspect 1 provides a method to isolate compounds having anti-tyrosinase activity, comprising:
   a) providing a cold press processed fruit seed extract dissolved or suspended in water, ethanol or a water-ethanol mix to provide a first mixture having solids and a liquid having compounds including ellagic acid or an ellagitannin; and
   b) separating the liquid from the solids to provide for a fraction that is enhanced in ellagic acid or ellagitannin and has anti-tyrosinase activity.
Aspect 2 provides a method to isolate ellagic acid or ellagitannin, comprising:
   a) providing a raspberry, blackberry or blueberry seed extract suspended in water, ethanol or a water-ethanol mix, thereby providing a mixture;
   b) incubating the mixture under conditions that allow for extraction of compounds including ellagic acid or ellagitannin; and
   c) separating the extracted compounds to provide for a fraction that is enhanced in ellagic acid or ellagitannin.
Aspect 3 provides the method of Aspect 1 or 2 wherein the method is free of the use of methanol.
Aspect 4 provides the method of Aspect 3 wherein the water-ethanol mix comprises 70:30 ethanol:water.
Aspect 5 provides the method of any one of Aspects 1 to 4 wherein the fraction inhibits eumelanin or pheomelanin production in human skin.
Aspect 6 provides the method of any one of Aspects 1 to 5 wherein the extract is a powder.
Aspect 7 provides the method of any one of Aspects 1 to 6 further comprising removing the solvent from the extracted compounds.
Aspect 8 provides the method of any one of Aspects 1 to 7 wherein separation further comprises:
   evaporating the extract solution to form a dry product;
   mixing the dry product with a 1:1 mixture of ethanol:acetone; and
   filtering the mixture of dry product.
Aspect 9 provides the method of any one of Aspects 1 to 7 wherein the separation further includes subjecting the mixture to separation on a C8 column, to separation on a C18 solid phase extraction column or to separation on an ion exchange chromatography column, or any combination thereof.
Aspect 10 provides the method of any one of Aspects 1 to 9 wherein separation further includes subjecting the mixture to high pressure liquid chromatography.
Aspect 11 provides the method of any one of Aspects 1 to 10 wherein the fruit is a red raspberry.
Aspect 12 provides the method of any one of Aspects 1 to 10 wherein the seed extract is a raspberry, blackberry, blueberry or pomegranate seed extract.
Aspect 13 provides a fraction obtained by the method of any one of Aspects 1 to 12.
Aspect 14 provides the use of the fraction of Aspect 13 in a dietary supplement, nutraceutical, or cosmetic formulation.
Aspect 15 provides the use of the fraction of Aspect 14 for skin lightening.
Aspect 16 provides a lotion, cream, solid, serum, or gel for preventing, inhibiting or treating hyperpigmentation of skin comprising an effective amount of the fraction of Aspect 13.
Aspect 17 provides a topical composition comprising an extract of raspberry seed, blackberry seed, blueberry seed or pomegranate seed having ellagic acid or ellagitannin.
Aspect 18 provides the composition of Aspect 17 further comprising a solvent.
Aspect 19 provides the composition of Aspect 18 wherein the solvent comprises an alcohol, oil or water.
Aspect 20 provides the composition of Aspect 18 wherein the solvent has a pH of about 1 to about 3.
Aspect 21 provides the composition of any one of Aspects 17 to 20 which is a lotion, serum, cream, solid or gel.
Aspect 22 provides the composition of any one of Aspects 17 to 21 wherein the extract is a raspberry seed extract.
Aspect 23 provides the composition of any one of Aspects 17 to 21 which has about 10 wt % to about 30 wt % of the extract.
Aspect 24 provides the composition of any one of Aspects 17 to 21 which has about 10 wt % to about 90 wt % of the extract.
Aspect 25 provides the composition of any one of Aspects 17 to 21 which has about 5 wt % to about 20 wt % of the extract.
Aspect 26 provides the composition of any one of Aspects 17 to 25 further comprising an emollient.
Aspect 27 provides the composition of Aspect 26 wherein the emollient is about 5 wt % to about 30 wt %, 10 wt % to about 30 wt %, 5 wt % to about 10 wt % or 1 wt % to about 5 wt % of the composition.
Aspect 28 provides the composition of any one of Aspects 17 to 27 further comprising a thickener.
Aspect 29 provides the composition of any one of Aspects 17 to 28 further comprising a preservative.
Aspect 30 provides the composition of Aspect 28 wherein the thickener is about 0.05 wt % to about 2 wt %.
Aspect 31 provides the composition of any one of Aspects 17 to 30 further comprising one or more emulsifiers.
Aspect 32 provides the composition of Aspect 31 wherein the emulsifier is about 1 wt % to about 5 wt % of the composition.
Aspect 33 provides the composition of any one of Aspects 17 to 32 further comprising a buffer.
Aspect 34 provides a topical liquid composition comprising an extract of raspberry seed, blackberry seed, blueberry seed or pomegranate seed having ellagic acid or ellagitannin and a solvent.
Aspect 35 provides the composition of Aspect 34 wherein the extract is a raspberry seed extract.
36 provides the composition of Aspect 34 or 35 which has about 10 wt % to about 30 wt %, about 5 wt % to about 20 wt %, or about 15 wt % to about 35 wt % of the extract.
Aspect 37 provides the composition of any one of Aspects 34 to 36 further comprising an emollient.
Aspect 38 provides the composition of Aspect 37 wherein the emollient comprises about 10 wt % to about 30 wt %, about 5 wt % to about 20 wt %, or about 15 wt % to about 35 wt %.
Aspect 39 provides the composition of Aspect 38 wherein the extract is a raspberry seed extract comprising about 10 wt % to about 30 wt and the solvent comprises ethanol.
Aspect 40 provides a topical serum composition comprising an extract of raspberry seed, blackberry seed, blueberry seed or pomegranate seed having ellagic acid or ellagitannin and a solvent.
Aspect 41 provides the composition of Aspect 40 wherein the extract is a raspberry seed extract.
Aspect 42 provides the composition of Aspect 40 or 41 which has about 10 wt % to about 30 wt %, 20 wt % to about 50 wt %, 50 wt % to about 90 wt %, or about 10 wt % to about 90 wt % of the extract.
Aspect 43 provides the composition of any one of Aspects 40 to 42 further comprising an emollient.
Aspect 44 provides the composition of Aspect 43 wherein the emollient comprises about 5 wt % to about 30 wt %, about 5 wt % to about 10 wt %, or about 15 wt % to about 35 wt % of the composition.
Aspect 45 provides the composition of Aspect 43 or 44 wherein the extract is a raspberry seed extract comprising about 10 wt % to about 90 wt % and the solvent comprises oil.
Aspect 46 provides the composition of Aspect 45 which further comprises an emollient at about 5 wt % to about 20 wt %, 5 wt % to about 30 wt % or about 10 wt % to about 40 wt %.
Aspect 47 provides the composition of Aspect 43 or 44 wherein the extract is a raspberry seed extract comprising about 10 wt % to about 30 wt % or 5 wt % to about 30 wt % and the solvent comprises water.
Aspect 48 provides the composition of Aspect 47 further comprising one or more emulsifiers.
Aspect 49 provides the composition of Aspect 47 wherein the emulsifier comprises about 1 wt % to about 5 wt %, about 2 wt % to about 6 wt %, or about 3 wt % to about 7 wt %.
Aspect 50 provides a topical oil in water emulsion composition comprising an extract of raspberry seed, blackberry seed, blueberry seed or pomegranate seed having ellagic acid or ellagitannin and a solvent.
Aspect 51 provides the composition of Aspect 50 wherein the extract is a raspberry seed extract.
Aspect 52 provides the composition of Aspect 50 or 51 which has about 10 wt % to about 30 wt %, or about 5 wt % to about 20 wt % of the extract.
Aspect 53 provides the composition of any one of Aspects 50 to 52 further comprising one or more emulsifiers.
Aspect 54 provides the composition of Aspect 53 wherein the one or more emulsifiers comprise about 2 wt % to about 5 wt %, about 4 wt % to about 10 wt %, or about 5 wt % to about 15 wt %.
Aspect 55 provides the composition of Aspect 54 which further comprises an emollient, a buffer, or both.
Aspect 56 provides the composition of any one of Aspects 50 to 55 wherein the solvent comprises water.
Aspect 57 provides a use of the composition of any one of Aspects 17 to 56 for skin lightening.

What is claimed is:

1. A method to isolate compounds having anti-tyrosinase activity, comprising:
a) providing a cold press processed fruit seed extract dissolved or suspended in water, ethanol or a water-ethanol mix to provide a first mixture having solids and a liquid having compounds including ellagic acid or an ellagitannin; and
b) separating the liquid from the solids to provide for a fraction that is enhanced in ellagic acid or ellagitannin and has anti-tyrosinase activity.

2. The method of claim 1 wherein the method is free of the use of methanol.

3. The method of claim 1 wherein the water-ethanol mix comprises 70:30 ethanol:water.

4. The method of claim 1 wherein the fraction inhibits eumelanin or pheomelanin production in human skin.

5. The method of claim 1 wherein the extract is a powder.

6. The method of claim 1 further comprising removing the solvent from the extracted compounds.

7. The method of claim 1 wherein the separation further comprises:
evaporating the extract solution to form a dry product;
mixing the dry product with a 1:1 mixture of ethanol:acetone; and
filtering the mixture of dry product.

8. The method of claim 1 wherein the separation further includes subjecting the mixture to separation on a C8 column, to separation on a C18 solid phase extraction column or to separation on an ion exchange chromatography column, or any combination thereof.

9. The method of claim 1 wherein separation further includes subjecting the mixture to high pressure liquid chromatography.

10. The method of claim 1 wherein the fruit is a red raspberry.

11. The method of claim 1 wherein the seed extract is a raspberry, blackberry, blueberry or pomegranate seed extract.

12. A method to isolate ellagic acid or ellagitannin, comprising:
a) providing a raspberry, blackberry or blueberry seed extract suspended in water, ethanol or a water-ethanol mix, thereby providing a mixture;
b) incubating the mixture under conditions that allow for extraction of compounds including ellagic acid or ellagitannin; and
c) separating the extracted compounds to provide for a fraction that is enhanced in ellagic acid or ellagitannin.

13. A topical composition comprising an extract of raspberry seed, blackberry seed, blueberry seed or pomegranate seed having ellagic acid or ellagitannin.

14. The composition of claim 13 which is a lotion, serum, cream, solid or gel.

15. The composition of claim 13 wherein the extract is a raspberry seed extract.

16. The composition of claim 13 which has about 10 wt % to about 30 wt % of the extract.

17. The composition of claim 13 further comprising an emollient.

18. The composition of claim 13 further comprising a thickener.

19. The composition of claim 13 further comprising a preservative.

20. The composition of claim 13, further comprising a water solvent.
